(19) United States
(12) Patent Application Publication
(10) Pub. No.: US 2005/0277579 A1
(43) Pub. Date: Dec. 15, 2005

(54) Compositions for affecting weight loss

(76) Inventors: Ranga Krishnan, Chapel Hill, NC (US); Kishore Gadde, Durham, NC (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/117,139

(22) Filed: Apr. 27, 2005

Related U.S. Application Data
(60) Provisional application No. 60/567,922, filed on May 3, 2004.

(51) Int. Cl. A61K 38/28; A61K 31/7008; A61K 31/4439; A61K 31/426; A61K 31/425; A61K 31/19

(52) U.S. Cl. 514/3; 514/243; 514/340; 514/369; 514/592; 514/635; 514/557; 514/23; 514/372

(57) ABSTRACT

Disclosed are compositions for affecting weight loss comprising a first compound and a second compound, where the first compound is an antidiabetic and the second compound is a anticonvulsant. Also disclosed are methods of affecting weight loss, increasing energy expenditure, increasing satiety in an individual, or suppressing the appetite of an individual, comprising identifying an individual in need thereof and treating that individual with an antidiabetic and an anticonvulsant.

### RELATED APPLICATIONS

This application claims priority to the U.S. Provisional Patent Application Ser. No. 60/567,922, entitled “COMPOSITIONS FOR AFFECTING WEIGHT LOSS,” and filed on May 3, 2004, by Ranga Krishnan, which is hereby incorporated by reference herein in its entirety.

### BACKGROUND OF THE INVENTION

1. Field of the Invention

The present invention is in the field of pharmaceutical compositions and methods for the treatment of obesity and for affecting weight loss in individuals.

2. Description of the Related Art

Obesity is a disorder characterized by the accumulation of excess fat in the body. Obesity has been recognized as one of the leading causes of disease and is emerging as a global problem. Increased instances of complications such as hypertension, non-insulin dependent diabetes mellitus, arteriosclerosis, dyslipidemia, certain forms of cancer, sleep apnea, and osteoarthritis have been related to increased instances of obesity in the general population.

Obesity has been defined in terms of body mass index (BMI). BMI is calculated as weight (kg)/[height (m)]². According to the guidelines of the U.S. Centers for Disease Control and Prevention (CDC), and the World Health Organization (WHO) (World Health Organization. Physical status: The use and interpretation of anthropometry. Geneva, Switzerland: World Health Organization 1995. WHO Technical Report Series), for adults over 20 years old, BMI falls into one of these categories: below 18.5 is considered underweight, 18.5-24.9 is considered normal, 25.0-29.9 is considered overweight, and 30.0 and above is considered obese.

Prior to 1994, obesity was generally considered a psychological problem. The discovery of the adipostatic hormone leptin in 1994 (Zhang et al., “Positional cloning of the mouse obese gene and its human homologue,” Nature 1994; 372:425-432) brought forth the realization that, in certain cases, obesity may have a biochemical basis. A corollary to this realization was the idea that the treatment of obesity may be achieved by chemical approaches. Since then, a number of such chemical treatments have entered the market. The most famous of these attempts was the introduction of Fen-Phen, a combination of fenfluramine and phentermine. Unfortunately, it was discovered that fenfluramine caused heart-valve complications, which in some cases resulted in the death of the user. Fenfluramine has since been withdrawn from the market. There has been some limited success with other combination therapy approaches, particularly in the field of psychological eating disorders. One such example is Devlin, et al., Int. J. Eating Disord. 28:325-332, 2000, in which a combination of phentermine and fluoxetine showed some efficacy in the treatment of binge eating disorders. Of course, this disorder is an issue for only a small portion of the population.

In addition to those individuals who satisfy a strict definition of medical obesity, a significant portion of the adult population is overweight. These overweight individuals would also benefit from the availability of an effective weight-loss composition. Therefore, there is an unmet need in the art to provide pharmaceutical compositions that can affect weight loss without having other adverse side effects.

### SUMMARY OF THE INVENTION

Disclosed are compositions for affecting weight loss comprising a first compound and a second compound, where the first compound is an antidiabetic and the second compound is an anticonvulsant.

### DETAILED DESCRIPTION OF THE PREFERRED EMBODIMENT

In a first aspect, the present invention is directed to a composition for the treatment of obesity or for affecting weight loss comprising a first compound and a second compound, where the first compound is an antidiabetic and the second compound is an anticonvulsant.

In certain embodiments the antidiabetic is effective in reducing the level of glucose in the blood of a mammal. In certain embodiments, the anticonvulsant is effective in reducing convulsions in a mammal. The mammal may be selected from the group consisting of mice, rats, rabbits, guinea pigs, dogs, cats, sheep, goats, cows, primates, such as monkeys, chimpanzees, and apes, and humans.

In some embodiments the antidiabetic is one of the following: a biguanide, glucosidase inhibitor, insulin, meglitinide, sulfonylurea, or a thiazolidinedione. An example of a biguanide is metformin hydrochloride. Examples of glucosidase inhibitors include acarbose and miglitol. Examples of insulin include human insulin, pork insulin, beef insulin, beef-pork insulin, insulin from different sources such as recombinant DNA and animal sources, as well as regular, NPH, and LENTE® types of insulin. Other examples of insulin include mixtures of the various forms of insulin (e.g. NPH and regular human and pork insulin). Other examples of insulin include mixtures of Insulin Lispro Protamine and Insulin Injection (rDNA origin), a 50/50 (or a 70/30) mixture of Human Insulin Isophane Suspension and Human Insulin Injection, a 70/30 mixture of NPH Human Insulin Isophane Suspension and Human Insulin Injection (rDNA), insulin glargine, insulin lispro, insulin aspart, as well as insulin mixed with other ingredients such as zinc crystals or in a phosphate buffer. Insulin may be from Saccharomyces cerevisiae or other sources. Examples of meglitinides include nateglinide and repaglinide. Examples of sulfonylureas include glimepiride, glyburide, glibenclamide, gliquidone, gliclazide, chlorpropamide, tolbutamide, tolazamide and glipizide. Examples of thiazolidinediones include rosiglitazone and pioglitazone. Also included are extended release formulations of the above drugs, as well as combinations of the above drugs and pharmaceutically acceptable salts or prodrugs thereof. In certain embodiments, the antidiabetic is metformin.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl) methylamine, and salts thereof with amino acids such as arginine, lysine, and the like.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug, or may demonstrate increased palatability or be easier to formulate. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to provide the active moiety.

In some embodiments, the second compound is an anticonvulsant. Examples of anticonvulsants include barbiturates, benzodiazepines, GABA analogues, hydantoins, miscellaneous anticonvulsants, phenyltriazines, and succinimides. An example of a barbiturate includes pentobarbital. Examples of benzodiazepines include clonazepam, clorazepate, and diazepam. Examples of GABA analogues include tiagabine, pregabalin and gabapentin. Examples of hydantoins include fosphenytoin, phenytoin, and 5,5-Diphenylhydantoin. Examples of miscellaneous anticonvulsants include carbamazepine, oxcarbazepine, valproate, valproic acid, divalproex, valrocemide, felbamate, levetiracetam, retigabine, lacosamide, talampanel, topiramate, and zonisamide. An example of a phenyltriazine is lamotrigine. Examples of succinimides include methsuximide and ethosuximide. Also included are extended release formulation of the above drugs, pharmaceutically acceptable salts, as well as combinations of the above drugs. In certain embodiments, the anticonvulsant is zonisamide, while in other embodiments, the anticonvulsant is topiramate.

In another aspect, the present invention relates to a method of affecting weight loss, comprising identifying an individual in need thereof and treating that individual with an antidiabetic and an anticonvulsant.

In certain embodiments, the individual has a body mass index (BMD) greater than 25. In other embodiments, the individual has a BMI greater than 30. In still other embodiments, the individual has a BMI greater than 40. However, in some embodiments, the individual may have a BMI less than 25. In these embodiments, it may be beneficial for health or cosmetic purposes to affect weight loss, thereby reducing the BMI even further.

In some embodiments, the treating step of the above method comprises administering to the individual a first compound and a second compound, where the first compound is an antidiabetic and the second compound is a anticonvulsant.

In some embodiments the first compound and the second compound are administered more or less simultaneously. In other embodiments the first compound is administered prior to the second compound. In yet other embodiments, the first compound is administered subsequent to the second compound.

In certain embodiments, the first compound and the second compound are administered individually. In other embodiments, the first compound and the second compound are covalently linked to each other such that they form a single chemical entity. The single chemical entity is then digested and is metabolized into two separate physiologically active chemical entities; one of which is the first compound and the other one is the second compound.

In another aspect, the present invention relates to a method of increasing satiety in an individual comprising identifying an individual in need thereof and treating that individual with a first compound and a second compound, where the first compound is an antidiabetic and the second compound is an anticonvulsant.

In some embodiments the first compound and the second compound are administered nearly simultaneously. In other embodiments the first compound is administered prior to the second compound. In yet other embodiments, the first compound is administered subsequent to the second compound.

In yet another aspect, the present invention relates to a method of suppressing the appetite of an individual comprising identifying an individual in need thereof and treating that individual by administering to the individual a first compound and a second compound, where the first compound is an antidiabetic and the second compound is a anticonvulsant.

In some embodiments the first compound and the second compound are administered nearly simultaneously. In other embodiments the first compound is administered prior to the second compound. In yet other embodiments, the first compound is administered subsequent to the second compound.

In another aspect, the present invention relates to a method of increasing energy expenditure in an individual comprising identifying an individual in need thereof and treating that individual by administering to the individual a first compound and a second compound, where the first compound is an antidiabetic and the second compound is a anticonvulsant.

In some embodiments the first compound and the second compound are administered nearly simultaneously. In other embodiments the first compound is administered prior to the second compound. In yet other embodiments, the first compound is administered subsequent to the second compound.

In certain embodiments disclosed herein, an individual is given a pharmaceutical composition comprising a combination of two or more compounds to affect weight loss. In some of these embodiments, each compound is a separate chemical entity. However, in other embodiments, the two compounds are joined together by a chemical linkage, such as a covalent bond, so that the two different compounds form separate parts of the same molecule. The chemical linkage is selected such that after entry into the body, the linkage is broken, such as by enzymatic action, acid hydrolysis, base hydrolysis, or the like, and the two separate compounds are then formed.

In another aspect, the invention relates to a pharmaceutical composition comprising a combination of an antidiabetic and an anticonvulsant, as described above, or comprising a linked molecule, as described herein, and a physiologically acceptable carrier, diluent, or excipient, or a combination thereof.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose or sublingual or by delivery through a skin patch or intranasal of between 0.1 mg and 5000 mg of each ingredient, preferably between 1 mg and 2500 mg, e.g. 25 to 2500 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of each ingredient up to 400 mg per day. Thus, the total daily dosage by oral administration of each ingredient will typically be in the range 1 to 2500 mg and the total daily dosage by parenteral administration will typically be in the range 0.1 to 400 mg. Suitably the compounds will be administered for a period of continuous therapy, for example for a week or more, or for months or years.

In one embodiment, the dosage range for metformin hydrochloride, for an oral dose, will vary between about 500 mg to about 2500 mg per day. In a preferred embodiment, the dosage range, for an oral dose will vary from about 500 mg three times a day to about 2500 mg a day.

In one embodiment, the dosage range for Zonisamide, for an oral dose, is in the range of about 25 to about 600 mg per day.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen that maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

It will be understood by those of skill in the art that numerous and various modifications can be made without departing from the spirit of the present invention. Therefore, it should be clearly understood that the forms of the present invention are illustrative only and are not intended to limit the scope of the present invention.

### SOME EMBODIMENTS OF THE INVENTION

Some of the embodiments of the present invention are as follows:

In the first embodiment, the invention relates to a composition for affecting weight loss comprising a first compound and a second compound, wherein said first compound is an antidiabetic and said second compound is an anticonvulsant.

In the second embodiment, the invention relates to the composition of the first embodiment, wherein said antidiabetic has antihyperglycemic characteristics in a mammal.

In the third embodiment, the invention relates to the composition of the first embodiment, wherein said antidiabetic is selected from the group consisting of biguanides, glucosidase inhbitors, insulins, meglitinides, sulfonylureas, thiazolidinediones, and pharmaceutically acceptable salts or prodrugs thereof.

In the fourth embodiment, the invention relates to the composition of the third embodiment, wherein the biguanide is metformin hydrochloride.

In the fifth embodiment, the invention relates to the composition of the third embodiment, wherein the glucosidase inhibitor is selected from the group consisting of acarbose, miglitol, and combinations thereof.

In the sixth embodiment, the invention relates to the composition of the third embodiment, wherein the insulin is selected from the group consisting of human, pork, beef, and combinations thereof.

In the seventh embodiment, the invention relates to the composition of the third embodiment, wherein the meglitinide is selected from the group consisting of nateglinide, repaglinide, and combinations thereof.

In the eighth embodiment, the invention relates to the composition of the third embodiment, wherein the sulfonylurea is selected from the group consisting of glimepiride, glyburide, glibenclamide, gliquidone, gliclazide, chlorpropamide, tolbutamide, tolazamide and glipizide, and combinations thereof.

In the ninth embodiment, the invention relates to the composition of the third embodiment, wherein the thiazolidinedione is selected from a group consisting of rosiglitazone and pioglitazone.

In the tenth embodiment, the invention relates to the composition of the first embodiment, wherein said second compound is selected from the group consisting of a barbiturate, a benzodiazepine, GABA analogue, hydantoins, miscellaneous anticonvulsant, phenyltriazine, a succinimide, pharmaceutically acceptable salts or prodrugs thereof, and combinations thereof.

In the eleventh embodiment, the invention relates to the composition of the tenth embodiment, wherein said barbiturate is pentobarbital.

In the twelfth embodiment, the invention relates to the composition of the tenth embodiment, wherein said benzodiazepine is selected from the group consisting of clonazepam, clorazepate, diazepam, and combinations thereof.

In the thirteenth embodiment, the invention relates to the composition of the tenth embodiment, wherein said GABA analogue is selected from the group consisting of tiagabine, pregabalin, gabapentin, and combinations thereof.

In the fourteenth embodiment, the invention relates to the composition of the tenth embodiment, wherein said hydantoin is selected from the group consisting of fosphenytoin, phenytoin, 5,5-Diphenylhydantoin, and combinations thereof.

In the fifteenth embodiment, the invention relates to the composition of the tenth embodiment, wherein said miscellaneous anticonvulsant is selected from the group consisting of carbamazepine, oxcarbazepine, valproate, valproic acid, divalproex, valrocemide, felbamate, lacosamide, talampanel, retigabine, levetiracetam, topiramate, zonisamide, and combinations thereof.

In the sixteenth embodiment, the invention relates to the composition of the tenth embodiment, wherein said phenyltriazine is lamotrigine.

In the seventeenth embodiment, the invention relates to the composition of the tenth embodiment, wherein said succinimide is selected from the group consisting of methsuximide, ethosuximide, and combinations thereof.

In the eighteenth embodiment, the invention relates to the composition of the first embodiment, wherein said first compound is an antihyperglycemic and said second compound is a zonisamide.

In the nineteenth embodiment, the invention relates to the composition of the first embodiment, wherein said first compound is metformin hydrochloride and said second compound is zonisamide.

In the twentieth embodiment, the invention relates to the composition of the first embodiment, wherein said first compound is topiramate and said second compound is zonisamide.

In the twenty first embodiment, the invention relates to the composition of the nineteenth embodiment, wherein the zonisamide is in a time-release formulation.

In the twenty second embodiment, the invention relates to a method of affecting weight loss, comprising identifying an individual in need thereof and treating that individual with an antidiabetic and an anticonvulsant.

In the twenty third embodiment, the invention relates to the method of the twenty second embodiment, wherein said individual has a body mass index greater than 25.

In the twenty fourth embodiment, the invention relates to the method of the twenty second embodiment, wherein the antidiabetic is selected from biguanides, glucosidase inhibitors, insulins, meglitinides, sulfonylureas, and pharmaceutically acceptable salts and prodrugs thereof.

In the twenty fifth embodiment, the invention relates to the method of the twenty second embodiment, wherein the antidiabetic is selected from the group consisting of metformin hydrochloride, acarbose, miglitol, human insulin, pork insulin, beef insulin, beef-pork insulin, insulin glargine, insulin lispro, insulin aspart, nateglinide, repaglinide, glimepiride, glyburide, chlorpropamide, tolazamide, glibenclamide; gliclazide, gliquidone, tolbutamide, glibenclamide, glipizide, extended release formulations of the above drugs, and combinations of the above drugs.

In the twenty sixth embodiment, the invention relates to the method of the twenty second embodiment, wherein the anticonvulsant is selected from the group consisting of barbiturates, benzodiazepines, GABA analogues, hydantoins phenyltriazines, and succinimides, and pharmaceutically acceptable salts or prodrugs thereof.

In the twenty seventh embodiment, the invention relates to the method of the twenty second embodiment, wherein the anticonvulsant is selected from the group consisting of pentobarbital, clonazepam, clorazepate, diazepam, tiagabine, gabapentin, pregabalin, fosphenytoin, phenytoin, phenytoin, 5,5-Diphenylhydantoin, carbamazepine, oxcarbazepine, valproate, valproic acid, divalproex, valrocemide, felbamate, levetiracetam, topiramate, zonisamide, lamotrigine, methsuximide, ethosuximide, retigabine, lacosamide, talampanel, extended release formulation of the above drugs, and combinations of the above drugs.

In the twenty eighth embodiment, the invention relates to the method of the twenty second embodiment, wherein said first compound and said second compound are administered nearly simultaneously.

In the twenty ninth embodiment, the invention relates to the method of the twenty second embodiment, wherein said first compound is administered prior to said second compound.

In the thirtieth embodiment, the invention relates to the method of the twenty second embodiment, wherein said first compound is administered subsequent to said second compound.

In the thirty first embodiment, the invention relates to a method of increasing satiety in an individual comprising identifying an individual in need thereof and treating that individual with a first compound and a second compound, wherein said first compound is an antidiabetic and said second compound is a anticonvulsant.

In the thirty second embodiment, the invention relates to the method of the thirty first embodiment, wherein said first compound and said second compound are administered nearly simultaneously.

In the thirty third embodiment, the invention relates to the method of the thirty first embodiment, wherein said first compound is administered prior to said second compound.

In the thirty fourth embodiment, the invention relates to the method of the thirty first embodiment, wherein said first compound is administered subsequent to said second compound.

In the thirty fifth embodiment, the invention relates to a method of increasing energy expenditure in an individual comprising identifying an individual in need thereof and treating that individual with a first compound and a second compound, wherein said first compound is an antidiabetic and said second compound is an anticonvulsant.

In the thirty sixth embodiment, the invention relates to the method of the thirty fifth embodiment, wherein said first compound and said second compound are administered nearly simultaneously.

In the thirty seventh embodiment, the invention relates to the method of the thirty fifth embodiment, wherein said first compound is administered prior to said second compound.

In the thirty eighth embodiment, the invention relates to the method of the thirty fifth embodiment, wherein said first compound is administered subsequent to said second compound.

In the thirty ninth embodiment, the invention relates to a method of suppressing the appetite of an individual comprising identifying an individual in need thereof and treating that individual with a first compound and a second compound, wherein said first compound is an antidiabetic and said second compound is an anticonvulsant.

In the fortieth embodiment, the invention relates to the method of the thirty ninth embodiment, wherein said first compound and said second compound are administered nearly simultaneously.

In the forty first embodiment, the invention relates to the method of the thirty ninth embodiment, wherein said first compound is administered prior to said second compound.

In the forty second embodiment, the invention relates to the method of the thirty ninth embodiment, wherein said first compound is administered subsequent to said second compound.

In the forty third embodiment, the invention relates to a method of affecting weight loss in an individual comprising identifying an individual in need thereof and treating that individual with a combination of metformin hydrochloride and zonisamide.

In the forty fourth embodiment, the invention relates to the method of the forty third embodiment, wherein the individual has a BMI greater than 30.

In the forty fifth embodiment, the invention relates to the method of the forty third embodiment, wherein the individual has a BMI greater than 25.

In the forty sixth embodiment, the invention relates to the method of the forty third embodiment, wherein the metformin is in a time-release formulation.

In the forty seventh embodiment, the invention relates to the method of the forty sixth embodiment, wherein the plasma concentration level of both metformin and zonisamide follow a similar concentration profile.

In the forty eighth embodiment, the invention relates to the method of the forty sixth embodiment, wherein the metformin and the zonisamide are administered substantially simultaneously.

In the forty ninth embodiment, the invention relates to the method of the forty sixth embodiment, wherein the metformin is administered prior to the zonisamide.

In the fiftieth embodiment, the invention relates to the method of the forty sixth embodiment, wherein the metformin is administered subsequent to the zonisamide.

### EXAMPLES

The examples below are non-limiting and are merely representative of various aspects of the invention.

### Example 1

### Combination of Zonisamide and Metformin

Individuals having a BMI of greater than 25 and fasting plasma glucose ≧100 mg/dL are identified. Each individual is instructed to take one 25 mg tablet of zonisamide on a daily basis, in addition to one 500 mg tablet of metformin hydrochloride on a daily basis.

The individuals are monitored for a period of months. It is recommended that the dosage be adjusted so that each individual loses weight at a rate of 10% of initial weight every 6 months. However, the rate of weight loss for each individual may be adjusted by the treating physician based on the individual's particular needs.

If the initial dosage is not effective, then the zonisamide dosage can be increased by approximately 20 mg per day, though never exceeding 600 mg total per day. Alternatively, or concurrently, the metformin hydrochloride dosage can be increased by 20 mg per day, though never exceeding 2500 mg total per day. If the initial dosage results in a more rapid weight loss than the above rate, the dosage of each of zonisamide or metformin can be reduced.

In some cases, it is beneficial to administer one dose of zonisamide per day in conjunction with two or three or more doses of metformin throughout the day. Metformin may also be in a time-release formulation where the dose is administered once a day, but metformin gradually enters the blood stream throughout the day, or in the course of a 12 hour period.

1. A composition for affecting weight loss comprising a first compound and a second compound, wherein said first compound is an antidiabetic and said second compound is an anticonvulsant.

2. The composition of claim 1, wherein said antidiabetic is selected from the group consisting of biguanides, glucosidase inhbitors, insulins, meglitinides, sulfonylureas, thiazolidinediones, and pharmaceutically acceptable salts or prodrugs thereof.

3. The composition of claim 2, wherein the biguanide is metformin or metformin hydrochloride.

4. The composition of claim 2, wherein the glucosidase inhibitor is selected from the group consisting of acarbose, miglitol, and combinations thereof.

5. The composition of claim 2, wherein the meglitinide is selected from the group consisting of nateglinide, repaglinide, and combinations thereof.

6. The composition of claim 2, wherein the sulfonylurea is selected from the group consisting of glimepiride, glyburide, glibenclamide, gliquidone, gliclazide, chlorpropamide, tolbutamide, tolazamide and glipizide, and combinations thereof.

7. The composition of claim 2, wherein the thiazolidinedione is selected from a group consisting of rosiglitazone and pioglitazone.

8. The composition of claim 1, wherein said second compound is selected from the group consisting of a barbiturate, a benzodiazepine, GABA analogue, hydantoins, miscellaneous anticonvulsant, phenyltriazine, a succinimide, pharmaceutically acceptable salts or prodrugs thereof, and combinations thereof.

9. The composition of claim 8, wherein said barbiturate is pentobarbital.

10. The composition of claim 8, wherein said benzodiazepine is selected from the group consisting of clonazepam, clorazepate, diazepam, and combinations thereof.

11. The composition of claim 8, wherein said GABA analogue is selected from the group consisting of tiagabine, pregabalin, gabapentin, and combinations thereof.

12. The composition of claim 8, wherein said hydantoin is selected from the group consisting of fosphenytoin, phenytoin, 5,5-Diphenylhydantoin, and combinations thereof.

13. The composition of claim 8, wherein said miscellaneous anticonvulsant is selected from the group consisting of carbamazepine, oxcarbazepine, valproate, valproic acid, divalproex, valrocemide, felbamate, lacosamide, talampanel, retigabine, levetiracetam, topiramate, zonisamide, and combinations thereof.

14. The composition of claim 8, wherein said phenyltriazine is lamotrigine.

15. The composition of claim 8, wherein said succinimide is selected from the group consisting of methsuximide, ethosuximide, and combinations thereof.

16. The composition of claim 1, wherein said first compound is an antihyperglycemic and said second compound is a zonisamide.

17. The composition of claim 1, wherein said first compound is metformin hydrochloride and said second compound is zonisamide.

18. The composition of claim 1, wherein said first compound is topiramate and said second compound is zonisamide.

19. A method of affecting weight loss, comprising identifying an individual in need thereof and treating that individual with an antidiabetic and an anticonvulsant.

20. The composition of claim 19, wherein the antidiabetic is selected from the group consisting of metformin hydrochloride, acarbose, miglitol, human insulin, pork insulin, beef insulin, beef-pork insulin, insulin glargine, insulin lispro, insulin aspart, nateglinide, repaglinide, glimepiride, glyburide, chlorpropamide, tolazamide, glibenclamide, gliclazide, gliquidone, tolbutamide, glibenclamide, glipizide, extended release formulations of the above drugs, and combinations of the above drugs.

21. The composition of claim 19, wherein the anticonvulsant is selected from the group consisting of pentobarbital, clonazepam, clorazepate, diazepam, tiagabine, gabapentin, pregabalin, fosphenytoin, phenytoin, phenytoin, 5,5-Diphenylhydantoin, carbamazepine, oxcarbazepine, valproate, valproic acid, divalproex, valrocemide, felbamate, levetiracetam, topiramate, zonisamide, lamotrigine, methsuximide, ethosuximide, retigabine, lacosamide, talampanel, extended release formulation of the above drugs, and combinations of the above drugs.

22. A method of affecting weight loss in an individual comprising identifying an individual in need thereof and treating that individual with a combination of metformin hydrochloride and zonisamide.
